(19) United States
(12) Patent Application Publication
(10) Pub. No.: US 2017/0112768 A1
(43) Pub. Date: Apr. 27, 2017

(54) DIRECTLY COMPRESSIBLE GRANULAR MICROCRYSTALLINE CELLULOSE BASED EXCIPIENT, MANUFACTURING PROCESS AND USE THEREOF

(71) Applicant: AVANTOR PERFORMANCE MATERIALS, LLC, Center Valley, PA (US)

(72) Inventors: Nandu DEORKAR, Cedar Knolls, NJ (US); James FARINA, Nazareth, PA (US); Liliana MIINEA, Whitehall, PA (US); Sameer RANDIVE, Thane-West (IN)

(73) Assignee: AVANTOR PERFORMANCE MATERIALS, LLC, Center Valley, PA (US)

(21) Appl. No.: 15/398,207

(22) Filed: Jan. 4, 2017

Related U.S. Application Data
(63) Continuation of application No. 12/619,259, filed on Nov. 16, 2009.
Application No. 12/619,259 is a continuation-in-part of application No. 12/600,369, filed on Dec. 9, 2010, now abandoned.
(60) Provisional application No. 61/116,025, filed on Nov. 19, 2008.
(60) Provisional application No. 60/978,866, filed on Oct. 10, 2007.

(30) Foreign Application Priority Data
Oct. 7, 2008 (US) PCT/US08/11555

(51) Int. Cl. A61K 9/16; A61K 31/192; B01J 2/04; A61K 9/28; B29C 43/00; A61K 9/20; A61K 31/40

(52) U.S. Cl. CPC A61K 9/1682; A61K 9/1652; A61K 9/16; A61K 9/20; A61K 9/2095; A61K 9/1635; A61K 31/192; A61K 31/40; A61K 9/2893; A61K 9/2054; A61K 9/2027; B29C 43/003; B01J 2/04; B29K 2001/00

(57) ABSTRACT

An improved excipient comprising substantially homogeneous particles of a compressible, high functionality granular microcrystalline cellulose based excipient is provided. The improved excipient comprises microcrystalline cellulose and a binder, and optionally a disintegrant, and is formed by spraying a homogeneous slurry of the components. The excipient provides enhanced flowability/good flow properties, excellent/high compactibility, and increased API loading and blendability as compared to the individual components, and as compared to conventional excipients formed from the same materials. The improved excipient has strong intraparticle bonding bridges between the components, resulting in a unique structural morphology including significant open structures or hollow pores. The presence of these pores provides a surface roughness that is the ideal environment for improved blending with an API.

### CROSS REFERENCE TO RELATED APPLICATIONS

This application claims the benefit of U.S. Provisional Patent Application No. 61/116025, filed Nov. 19, 2008, and is a continuation-in-part of U.S. application Ser. No. 12/600,369 filed Nov. 16, 2009, which is the National U.S. application of PCT/US2008/011555 filed Oct. 7, 2008, which claims the benefit of U.S. Provisional Patent Application No. 60/978,866 filed Oct. 10, 2007.

### BACKGROUND OF INVENTION

The most commonly employed means to deliver drug substances is the tablet, typically obtained through the compression of appropriately formulated excipient powders. Tablets should be free of defects, have the strength to withstand mechanical shocks, and have the chemical and physical stability to maintain physical attributes over time and during storage. Undesirable changes in either chemical or physical stability can result in unacceptable changes in the bioavailability of the drug substance. In addition, tablets must be able to release the drug substance in a predictable and reproducible manner. The present invention relates to a novel excipient for use in the manufacture of pharmaceutical solid dosage forms such as tablets. The novel excipient is advantageously combined with at least one drug substance, hereinafter active pharmaceutical ingredient (API), and formed into tablets using a direct compression manufacturing method.

In order to successfully form tablets, the tableting mixture must flow freely from a feeder hopper into a tablet die, and be suitably compressible. Since most APIs have poor flowability and compressibility, APIs are typically mixed with varying proportions of various excipients to impart desired flow and compressibility properties. In typical practice, a compressible mixture is obtained by blending an API with excipients such as diluents/fillers, binders/adhesives, disintegrants, glidants/flow promoters, colors, and flavors. These materials may be simply blended, or may be wet or dry granulated by conventional methods. Once mixing is complete, a lubricating excipient is typically added and the resulting material compressed into tablets.

Unfortunately, there are few general rules regarding excipient compatibility with particular APIs. Therefore, when developing tablet formulations to meet particular desired characteristics, pharmaceutical scientists typically must conduct an extensive series of experiments designed to determine which excipients are physically and chemically compatible with a specific API. Upon completion of this work, the scientist deduces suitable components for use in one or more trial compositions.

Two conventional methods of making tablets are dry blending followed by direct compression, and granulation followed by direct compression. In a typical direct compression process, the API is blended with the desired excipients such as diluent/filler, binder, disintegrant, glidant, and colors. Once blending is complete a lubricating excipient is added and the resulting material is compressed into tablets.

The direct compression method is limited by and dependent on the specific API properties, and further upon the combination of the various excipients. Therefore granulation of the excipients with the API is typically employed in order to achieve satisfactory tablets and/or improve tablet production speed. Traditional methods of granulation include dry granulation, wet granulation, and spray granulation. Each of these methods has limitations regarding the particles produced from the process.

The dry granulation method consists of mixing the components to form a blend which is roll compacted. This process is limited as the particles are not held together strongly and easily fall apart. Roll compaction processing also results in reduction of compactibility of many excipients.

Wet granulation is a process in which excipients are bound together in the presence of a liquid binder in a blender system, to produce a wet granular blend which is dried. Spray granulation is a process in which excipients are bound together in a fluidized bed. These processes are batch processes, which limits production speed, and can produce a variable product.

These conventional processes are utilized to produce particles with improved powder flow characteristics to produce tablets having improved physical characteristics. However, these processes are time consuming and may not be compatible with many APIs.

Various attempts have been made to produce improved excipients. U.S. Pat. No. 4,675,188 to Chu et al. discloses a granular directly compressible anhydrous dicalcium phosphate excipient which purports to have a particle size sufficient for efficient direct compression tableting. According to the disclosure, dicalcium phosphate is dehydrated, and then granulated with a binder. The resulting product is purportedly a granular anhydrous dicalcium phosphate, characterized in that at least 90 percent of the particles are larger than 44 microns. This granular product purports to improve over commonly used precipitated anhydrous dicalcium phosphate, which is a fine, dense powder that must be agglomerated with a binder such as starch before it can be used in direct compression tableting. The process disclosed in this patent consists of coating anhydrous calcium phosphate with starch or another binder, purportedly resulting in binding of calcium phosphate particles to each other forming large particles. However, this granulated product is not a universal excipient, in that it lacks other necessary excipients, such as disintegrants, that are necessary to produce a pharmaceutically acceptable tablet after compression.

U.S. Pat. No. 6,746,693 discloses an agglomerated microcrystalline cellulose blend containing silicon dioxide, purported to have improved compressibility. The disclosure states that silicon dioxide is a critical component to improve compressibility. The two step process described includes spray granulation followed by wet granulation, and does not provide a complete universal excipient.

A commercially available excipient, Ludipress®, is disclosed in EP 0192173B1. Ludipress® is composed of lactose, crospovidone, and povidone. Lactose is known to have better flowability than microcrystalline cellulose due to inherently different particle shape and morphology. Lactose and povidone are water soluble components that mix well with a third non-water soluble component for granulation by spray drying. There is no disclosure of a complete universal excipient including two or more insoluble components, or a specific particle morphology to enable increased flowability, compactibility with various APIs and varying degrees of loading.

There exists therefore a need in the pharmaceutical industry for a complete and universal directly compressible granular excipient that consists of not only filler but also a binder and a disintegrant. The desired excipient is also compatible with a wide range of APIs, and has a particle shape, size, and morphology to provide optimal flowability and compressibility. This improved excipient would simplify tableting and may require one step mixing of the API and lubricant beibre direct compression.

There further exists a need in the pharmaceutical industry for a complete and universal directly compressible high functionality granular excipient that consists of a filler and a binder, but does not include a disintegrant. This excipient would have the advantage of being suitable for both dry and wet granulation, while regular excipients, such as microcrystalline cellulose, lose compressibility when wet granulated.

### SUMMARY OF INVENTION

An illustrative aspect of the present invention is a composition comprising about 75% to about 98% microcrystalline cellulose, about 1% to about 10% at least one binder, and about 1% to about 20% at least one disintegrant, wherein the microcrystalline cellulose, binder and disintegrant are indistinguishable when viewed with a SEM, thereby forming substantially homogeneous, substantially spherical particles.

Another illustrative aspect of the present invention is an excipient comprising about 75% to about 98% microcrystalline cellulose, about 1% to about 10% at least one binder, and about 1% to about 20% at least one disintegrant, wherein the excipient is formed by spraying an aqueous slurry comprised of the microcrystalline cellulose, binder and disintegrant.

Yet another illustrative aspect of the present invention is a method of making an excipient. The method comprises mixing a microcrystalline cellulose slurry with a disintegrant slurry to form a microcrystalline cellulose/disintegrant slurry; mixing a binder in water to form a viscous binder slurry; homogenizing the binder slurry with the microcrystalline cellulose/disintegrant slurry form a homogenized slurry; and spray dry granulating the homogenized slurry to form substantially homogeneous, substantially spherical particles of excipient.

A further illustrative aspect of the present invention is a pharmaceutical tablet comprising at least one active pharmaceutical ingredient and an excipient. The excipient comprises substantially homogeneous, substantially spherical particles including microcrystalline cellulose, at least one binder, and at least one disintegrant.

Yet a further illustrative aspect of the present invention is a method of making a pharmaceutical tablet. The method comprises mixing at least one active pharmaceutical ingredient and an excipient and compressing the resulting mixture to form a tablet. The excipient comprises substantially homogeneous, substantially spherical particles including microcrystalline cellulose, at least one binder, and at least one disintegrant.

An alternate illustrative aspect of the present invention is a composition comprising substantially homogeneous particles including about 90% to about 99% microcrystalline cellulose and about 1% to about 10% at least one binder.

Another alternate illustrative aspect of the present invention is an excipient comprising about 95% to about 99% microcrystalline cellulose and about 1% to about 5% at least one binder, wherein the excipient is formed by spray dry granulating an aqueous slurry comprised of the microcrystalline cellulose and binder.

Yet another alternate illustrative aspect of the present invention is a method of making an excipient. The method comprises mixing a binder in water to form a viscous solution, homogenizing microcrystalline cellulose into the viscous solution to form a slurry; and spraying the slurry to form substantially homogeneous particles of excipient.

Still another alternate illustrative aspect of the present invention is another method of making an excipient. The method comprises dissolving hydroxypropyl methylcellulose in water to form a viscous solution; mixing and homogenizing microcrystalline cellulose into the viscous solution to form a slurry; and spraying the slurry to form. substantially homogeneous particles.

A further alternate illustrative aspect of the present invention is a pharmaceutical tablet comprising at least one active pharmaceutical ingredient, a disintegrant and an excipient. The excipient comprises substantially homogeneous particles including microcrystalline cellulose and at least one binder.

Yet a further alternate illustrative aspect of the present invention is a method of making a pharmaceutical tablet. The method comprises mixing at least one active pharmaceutical ingredient, a disintegrant and an excipient and compressing the resulting mixture to form a tablet. The excipient comprises substantially homogeneous particles including microcrystalline cellulose and at least one binder.

### BRIEF DESCRIPTION OF:DRAWINGS

FIG. 1 is an illustration of SEM micrographs of the improved excipient of the present invention produced according to Example 1.

FIG. 2 is an illustration of SEM micrographs of the improved excipient of the present invention produced according to Example 2.

FIG. 3 is an illustration of SEM micrographs of microcrystalline cellulose.

FIG. 4 is an illustration of SEM micrographs of a commercially available excipient, Prosolv®90.

FIG. 5 is an illustration of SEM micrographs of a commercially available excipient, Ludipress®

FIG. 6 is an illustration of SEM micrographs of an excipient manufactured by conventional high shear wet granulation method according to Example 4.

FIG. 7 is an illustration of a flowability index comparison of an excipient made by conventional high shear wet granulation according to example 4 and the improved excipient of the present invention produced according to Examples 1, 2 and 3.

FIG. 8 is an illustration of SEM micrographs of multiple samplings of the improved excipient of the present invention produced according to Example 3.

FIG. 9 is an illustration of the dissolution profile for 62.5% Ibuprofen/Example 1 excipient/silica/magnesium stearate tablets.

FIG. 10 is an illustration of the effect of compression force on tablet hardness and tablet disintegration time for tablets pressed according to Example 21.

FIG. 11 is an illustration oldie effect of variable tonnage on tablet hardness for tablets pressed according to Example 21.

FIG. 12 is an illustration of SEM micrographs of multiple samplings of the alternate improved excipient of the present invention produced according to Example 22.

FIG. 13 is an illustration of SEM micrographs of multiple samplings of the alternate improved excipient of the present invention produced according to Example 23.

FIG. 14 is an illustration of SEM micrographs of MCC (98%)-HPMC (2%) prepared by high shear wet granulation (HSWG) according to Example 24.

### DETAILED DESCRIPTION

There is provided an excipient comprising substantially homogeneous, substantially spherical particles of a highly compressible granular microcrystalline cellulose based excipient, herein denoted the “improved excipient.” As defined herein, the term ‘substantially homogenous particles’ is defined as a composition in which the individual components of the composition are not individually distinguishable when viewed under SEM. The improved excipient provides enhanced flowability/good flow properties, excellent/high compactibility, and increased API loading and blendability as compared to the individual components, and as compared to conventional excipients formed from the same materials.

The improved excipient has strong intraparticle bonding bridges between the components, resulting in a unique structural morphology including significant open structures or hollow pores. The presence of these pores provides a surface roughness that is the ideal environment for improved blending with an API. Excellent blendability is an essential characteristic of an excipient as it allows tablets to be produced that contain a uniform amount of the API. Additionally,this improved excipient includes the necessary excipients, except for the optional lubricant, that are required to produce a pharmaceutically acceptable tablet.

The improved excipient is engineered to have particle size that results in the excipient being directly compressible, complete, and universal excipient for making pharmaceutical tablets. The excipient is considered complete since it includes a diluent, a binder and a disintegrant, and universal since it is surprisingly compatible with a variety of APIs. The components and physical characteristics of the improved excipient were carefully chosen and optimized to ensure its use in formulating a wide range of APIs.

The universality of this excipient overcomes the need for the traditional time consuming approach to formulation development, wherein the scientist develops a custom blend of various excipients to optimize flowability and compressibility for the particular API. It was unexpectedly discovered that the disclosed composition and process of making the improved excipient provides a substantially homogeneous, strong spherical particle having high increased porosity that provides good flowability and high compactibility. The improved excipient typically has an aerated bulk density of about 0.1-0.4 g/cc.

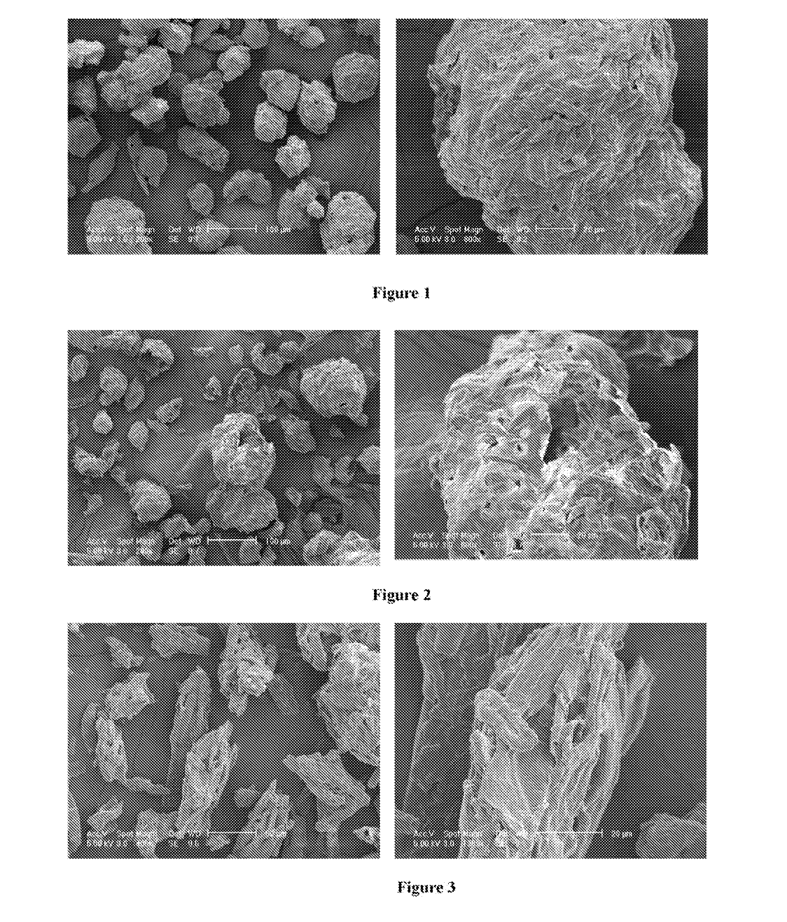

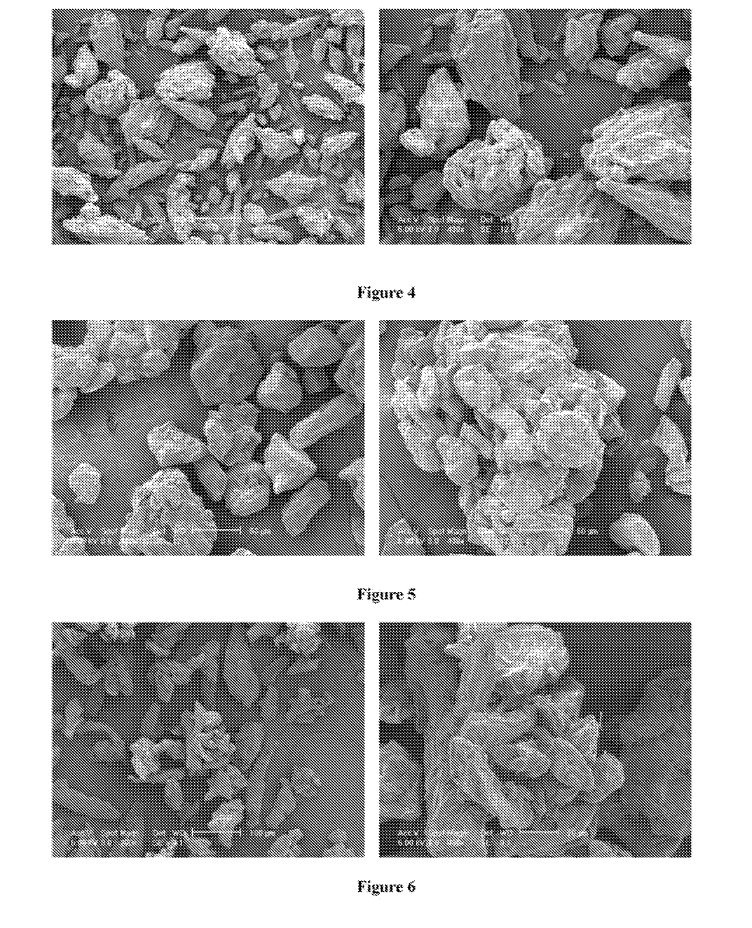

Unprocessed microcrystalline cellulose (MCC) has a needle-like shape when viewed under SEM (as illustrated in FIG. 3). The particle morphology of the improved excipient disclosed herein is unexpectedly unique as a substantially homogeneous spherical structure with holes or pores and hollow portions in the particles that can improve API loading capacity. As is illustrated in FIGS. 1 and 2, the term substantially homogeneous is meant herein to denote a structure in which the individual components cannot be distinguished under SEM scan. This contrasts with traditional excipients such as Prosolv® (as illustrated in FIG. 4) and Ludipress® (as illustrated in FIG. 5). These conventionally produced excipients do not produce the substantially homogeneous particle morphology of the improved excipient, but instead are composed of easily distinguished, agglomerated particles bonded together. The granules formed in the traditional and other disclosed processes are seen as a simple bonding of particles into irregularly shaped granules produced by agglomeration of distinct particles. It is common for these agglomerated particles to separate into the distinct components during transport or rough handling. The continuous spherical particles of the improved excipient, while including hollow portions, are unexpectedly robust and are not friable during handling and processing.

In the present invention, MCC is processed in combination with a polymeric binder and a cross-linked hygroscopic polymer to produce spherical particles having high porosity and strong intraparticle binding. The polymeric binder is selected from the class of cellulosic polymers or organic synthetic polymers having thermal stability at about 80° C. to about 120° C., dynamic viscosity in the range of about 2 mPa to about 50 mPa for a water solution of about 0.5% to about 5% wt/vol, water solubility in the range of about 0.5% to about 5% wt/vol and providing a surface tension in the range of about 40 dynes/cm to about 65 dynes/cm for about 0.5% to about 5% wt/vol water solution. Preferred binders from this class include hydroxypropyl methylcellulose, hydroxyethyl cellulose, hydroxypropyl cellulose, sodium carboxymethyl cellulose, and polyvinyl alcohol-polyethylene glycol graft copolymer and vinylpyrrolidone-vinyl acetate copolymer. Presently preferred is hydroxypropyl methylcellulose (HPMC). The cross-linked hygroscopic polymer disintegrant is preferably crospovidone (CPVD). As is seen in FIGS. 1 and 2, the processed particles are a substantially homogeneous composition of spheres with porous portions leading to at least partially hollow portions of the spheres. The granules are produced by the actual physical binding of the slurry mixture that becomes distinct particles when ejected out of the nozzle. The porosity and hollow portions result in improved API loading and blendability.

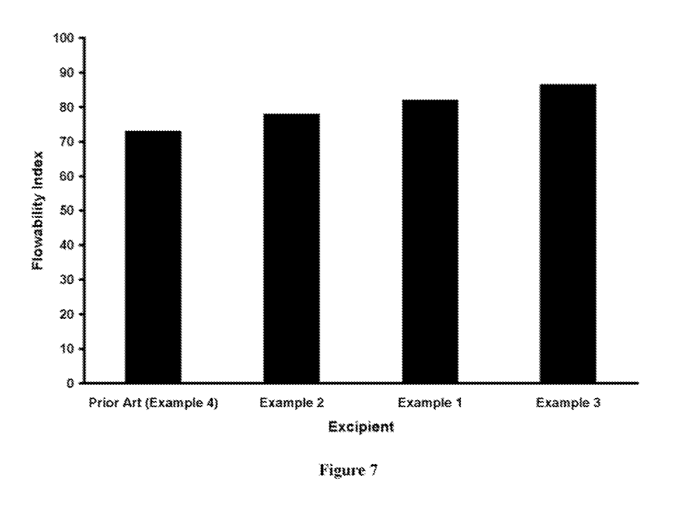

The improved excipient has excellent flowability. In general, when particle flow is poor, additional glidants such as silicon dioxide are added to improve flow. If the powder flow is not sufficient, poor tablet productivity will result. Characterization of the improved excipient particles by the Carr method, well know in art, showed a flowability index that exceeds 80, where a flowability index over 70 indicates good flowability. As is seen in Example 6, a Hosokawa powder tester, a test instrument that measures powder characteristics using a set of automated tests using the Carr method was used to determine that the improved excipient of Example 1 has a flowability index of 82. FIG. 7 illustrates a comparison of flowability index for a conventionally prepared excipient according to Example 4 with the improved excipient of the present invention.

As illustrated in Example 5, the granules of the material produced according to the invention are stronger than those of a similar material produced by a traditional high shear wet granulation process.

As illustrated in Examples 13 and 15, the improved excipient of the present invention produced acceptable tablets by direct compression when directly mixed with as low as about 1% API or as high as about 50% API. This indicates universal application and use of the material produced according to this invention. The use of greater than about 50% API may be accomplished by the use of a glidant component in the composition.

The process disclosed herein is a novel form of the spray drying granulation process. The new process consists of the homogenization of all three components of the excipient in the presence of water to create a slurry of the components. In one non-limiting, illustrative embodiment, a slurry of MCC is mixed with a slurry of cross-linked polyvinylpyrrolidone slurry to form a MCC/cross-linked polyvinylpyrrolidone slurry. Hydroxypropyl methylcellulose is then mixed with water to form a viscous hydroxypropyl methylcellulose slurry. The hydroxypropyl methylcellulose slurry is then mixed/homogenized with the MCC/cross-linked polyvinylpyrrolidone slurry to form a homogenized slurry. The homogenized slurry is then spray dry granulated to form substantially homogeneous, substantially spherical particles of excipient.

The homogenization process is carried out to bring the two insoluble components, MCC and a disintegrant, in contact with each other arid bound in close association with a viscous binder solution, for example hydroxypropyl methylcellulose. The evaporation of water at a high rate at high temperatures of 120° C. or more and the local action of HPMC holding all components together produces particle with unique shape and morphology.

In contrast, a traditional spray drying method uses compositions of one or two soluble components. Example 4, FIG. 6 illustrates the composition components of the present invention processed by the traditional wet granulation method. The material produced from the conventional high shear wet granulation process consisted of needle like friable particles that did not perform as well as the product formed by the present method, as illustrated in Examples 1 and 3. Compressiblity decreased, resulting in a 1.8 times decrease in the hardness of the placebo tablets pressed front the conventionally produced material as compared to the improved according to Example 1, see Example 7. The particle morphology is composed of irregular particles bonded together by simple intergranular bridges, as seen in FIG. 6.

The components of the improved excipient are processed by an improved wet homogenization/spray dry granulation method. In this process, a slurry is formed of two water insoluble components (typically with a large difference in composition between the two water insoluble components) and a third water soluble component. The resulting slurry is granulated to a desired particle size, typically greater than about 50 μm, preferably about 50 μm to about 250 μm, and more preferably about 90 μm to about 150 μm.

The excipient is formed by processing, or homogenizing, MCC with the polymeric binder and a cross-linked hygroscopic polymer disintegrant. In an illustrative embodiment, the excipient is formed from about 75% to about 98% MCC, in combination with about 1% to about 10% binder and about 1% to about 20% disintegrant. In a preferred embodiment, the excipient is formed from about 80% to about 90% MCC, about 2% to about 8% binder and about 3% to about 12% disintegrant. In a more preferred embodiment, the excipient is formed from about 85% to about 93%, about 2% to about 5% binder and about 10% disintegrant.

It has further been determined that varying the ratio of MCC and disintegrant to the binder affects the density of the final excipient. In an illustrative example, utilizing HPMC as the binder 5.5% HPMC yields an excipient with an aerated bulk density of 0.2 g/cc, see Example 2, wherein 2% HPMC yields an excipient with an aerated bulk density of 0.3 g/cc, see Example 1. The increase in bulk density indicates a lower porosity.

The use of the improved excipient will reduce formulation development to a series of blending steps: blending of an API with the improved excipient (which contains the essential components of tablet formulation, diluent, binder and disintegrant) and optionally a lubricant. The blending process will typically be followed by pressing high quality tablets by direct compression, for example by a rotary tabletting machine.

The “active ingredient” or “active agent”, referred herein as the API, refers to one or more compounds that have pharmaceutical activity, including therapeutic, diagnostic or prophylactic utility. The pharmaceutical agent may be present in an amorphous state, a crystalline state or a mixture thereof. The active ingredient may be present as is, taste masked, coated for enteric or controlled release. There is no limitation to the active pharmaceutical ingredient (API) that can be used with the present invention except that in which the API is incompatible with the microcrystalline cellulose.

Illustrative suitable active ingredients that can be used with the present invention include, but are not limited to: Antiviral agents, including but not limited to acyclovir, famciclovir; anthelmintic agents, including but not limited to albendazole; lipid regulating agents, including but not limited to atorvastatin calcium, simvastatin; angiotensin converting enzyme inhibitor including but not limited to benazepril hydrochloride, fosinopril sodium; angiotensin II receptor antagonist including but not limited to irbesartan, losartan potassium, valsartan; antibiotic including but not limited to doxycycline hydrochloride; antibacterial including but not limited to linezolid, metronidazole, norfloxacin; antifungal including but not limited to terbinafine; antimicrobial agent including but not limited to ciprofloxacin, cefixime; antidepressant, including but not limited to bupropione hydrochloride, fluoxetine; anticonvulsant including but not limited to carbamazepine; antihistamine including but not limited to loratadine; antimalarial including but not limited to mefloquine; antipsychotic agent including but not limited to olanzapine; anticoagulant including but not limited to warfarin; β-andrenergic blocking agent including but not limited to carvedilol, propranolol; selective H₁-receptor antagonist including but not limited to cetirizine hydrochloride, fexofenadine; histamine H₂-receptor antagonist including but not limited to cimetidine, famotidine, ranitidine hydrochloride, ranitidine; anti anxiety agent including but not limited to diazepam, lorazepam; zepam; anticonvulsants including but not limited to divalproex sodium, lamotrigine; inhibitor of steroid Type II 5α-reductase including but not limited to finasteride; actetylcholinesterase inhibitor including but not limited to galantamine; blood glucose lowering drug including but not limited to glimepiride, glyburide; vasodilator including but not limited to isosorbide dinitrate; calcium channel blocker including but not limited to nifedipine; gastric acid secretion inhibitor including but not limited to omeprazole; analgesic/antipyretics including but not limited to aspirin, acetaminophen, ibuprofen, naproxen sodium, oxycodone; erectile dysfunction including but not limited to sildenafil; diuretic including but not limited to hydrochlorothiazide; vitamins including but not limited to vitamin A, vitamin B1, vitamin B2, vitamin B6, vitamin B12, vitamin C, vitamin D, vitamin E, vitamin K or folic acid.

Illustrative, non-limiting examples of tablet formulations including an API can be found in the Examples, specifically acetaminophen, Examples 10-44; ibuprofen, example 16; naproxen sodium, Example 15; and atorvastatin calcium, Example 21.

The tablets produced utilizing the improved excipient of the present invention may include further additives and/or fillers as is known in the art. These addition components include but are not limited to excipients such as diluents/fillers, binders/adhesives, disintegrants, glidants/flow promoters, colors, and flavors. Illustrative Examples of tablet formulations of various weights, punches and embossing are shown in Example 18; coated tablets in Example 19; and tablets includingtillers in Example 20.

Therefore, the composition and processing steps disclosed herein produce an improved excipient exhibiting novel final particle morphology and unexpectedly improved compressibility.

In an alternate embodiment, the improved excipient is formulated from MCC and a binder, without a disintegrant (hereinafter the ‘alternate improved excipient’) It was unexpectedly discovered that the alternate improved excipient, comprised of MCC and at least one binder and formed according to the present invention, provides better flowability and higher compactibility than various grades of MCC. Moreover, the alternate improved excipient typically has an aerated bulk density of about 0.2 to 0.3 g/cc, and spherically shaped particles that have a roughness associated with them that allows better API blendability than various grades of MCC. This alternate improved excipient is suitable for both dry and wet granulation. When wet granulated the alternate improved excipient does not lose compressibility as compared with various grades of MCC which typically lose compressibility upon wet granulation.

The alternate improved excipient is produced as described above, without the addition of a disintegrant. In a preferred embodiment, the alternate improved excipient comprises about 90% to about 99% MCC and about 1% to about 10% binder; in a more preferred embodiment the alternate improved excipient comprises about 95% to about 99% MCC and about 1% to about 5% binder; and in a most preferred embodiment the alternate improved excipient comprises about 97% to about 99% MCC and about 1% to about 3% binder.

Examples 22 and 23 illustrate methods of making the alternate improved excipient, utilizing 98% MCC/2% HPMC and 95% MCC/5HPMC, respectively, utilizing a homogenization/spray dry granulation method. Examples 24, 25 and 26 illustrate methods of making the alternate improved excipient, utilizing 98% MCC/2% HPMC, 95% MCC/5HPMC, and 90% MCC/10% HPMC, respectively, utilizing a conventional wet granulation method, high shear wet granulation. Example 27 discloses the production of a prior art formulation, a powdered blend of MCC and HPMC. Examples 28 through 39 illustrate comparative testing of the alternate improved excipient and commercially available MCC. As is demonstrated in the examples, the alternate improved excipient provides homogeneous spherical granules with an average particle size of 100-150 microns. The alternate improved excipient has better flowability than various grades of MCC and due to the roughness associated to its particles has a better blend-ability with APIs. The alternate embodiment excipient granules are hard and do not break when tested for friability as compared to granules of similar composition prepared by HSWG. The alternate embodiment excipient does not lose compressibility when wet granulated as compared to MCC.

### EXAMPLE 1

### Preparation of Microcrystalline Cellulose—2% Hydroxypropyl Methylcellulose—Crospovidone Excipient According to the Present Invention

The improved excipient consists of microcrystalline cellulose at 85%, hydroxypropyl methyl cellulose at 2%, and crospovidone at 13%. The excipient was produced by a wet homogenization/spray dry granulation process. The apparatus used for the production of the excipient was a Co-current atomizer disc type with the disc RPM between 12000 and 25000 and the inlet temperatures of 180-250° C. Powdered MCC was converted into a slurry in a mixing chamber with deionized water to give a concentration of 23.3%. The other components, HPMC and crospovidone were also converted to a slurry with deionized water in a separate mixing chamber at 60° C. to a concentration of 5.9%. The MCC slurry was then transferred to the chamber containing the HPMC/crospovidone slurry and homogenized into a uniform mixture at 40-60° C. for 1 hour using circulating shear pump and an agitator to keep solids suspended in the solution thereby forming a uniform slurry. The slurry mixture was then spray dried through a rotary nozzle at a motor frequency of 33 Hz in the presence of hot air at an outlet temperature of 106-109° C. This constitutes the granule formation step. The fines were removed in a cyclone and the final product was collected to give the new improved excipient. SEM micrographs of the excipient of Example 1 are seen in FIG. 1. Unless otherwise noted, all SEM micrographs herein were recorded using a FE1 XL30 ESEM (environmental scanning electron microscope), voltage 5 kV, spot size 3, SE detector. The samples were sputtered with Iridium before SEM analysis (sputtering time 40 sec.)

The compressibility, aerated hulk density and tapped bulk density of the granular material were measured using a Powder Tester (Hosokawa Micron Corporation) Model PT-S. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm. D50 value was calculated based on the data collected in a “particle size distribution” measurement. An Air Jet Sieving instrument (Hosokawa Micron System) was used to determine the particle size distribution of the granular material. A set of four sieves (270 mesh, 200 mesh, 100 mesh and 60 mesh) was used. The sieving time for each sieve was 60 sec, while the vacuum pressure was maintained at 12-14 in. H₂O. The sample size was 5 g.

The “loss on drying” (LOD) value was determined using a Mettler Toledo Infrared Dryer LP16. The set temperature was 120° C. and the analysis was stopped When. constant weight was reached.

TABLE 1

| Powder Characteristics | Value |
|---|---|
| 1. Compressibility | 16.1% |
| 2. D50 | 113 um |
| 3. Aerated bulk density | 0.29 g/cc |
| 4. Tapped bulk density | 0.35 g/cc |
| 5. LOD | 3.0% |

### EXAMPLE 2

### Preparation of Microcrystalline Cellulose—5.5% Hydroxypropyl Methylcellulose—Crospovidone Excipient According to the Present Invention

The excipient consists of microcrystalline cellulose at 85.5%, hydroxypropyl methyl cellulose at 5.5%, and crospovidone at 9%. The excipient was produced by a wet homogenization/spray drying granulation process. The apparatus used for the production of the excipient is a Co-current atomizer disc type with the disc RPM between 12000-2.5000 and the inlet temperatures of 180-250 ° C. After granulation a cyclone separation device was used to remove the fines. Powdered MCC was converted into a slurry using deionized water in a mixing chamber to reach a concentration of 25.1%. The other components HPMC and crospovidone were first dry mixed and then also converted into a slurry with deionized water in a separate mixing chamber to a concentration of 11.4%. The MCC slurry was then transferred to the chamber containing the HPMC/crospovidone slurry and homogenized into a uniform mixture at 40-60° C. for 1 hour using circulating shear pump and an agitator to keep solid suspended in the solution to form uniform slurry The slurry mixture was then spray dried through a rotary nozzle at the motor frequency of 40.1 Hz in the presence of hot air at an outlet temperature of 106-109° C. This constitutes the granule formation step. The fines were removed in a cyclone and the final product was collected, see FIG. 2.

The powder characteristics were determined as described in example 1.

TABLE 2

| Powder Characteristics | Value |
|---|---|
| 1. Compressibility | 19.7% |
| 2. D50 | 104 um |
| 3. Aerated bulk density | 0.20 g/cc |
| 4. Tapped bulk density | 0.25 g/cc |
| 5. LOD | 2.0% |

### EXAMPLE 3

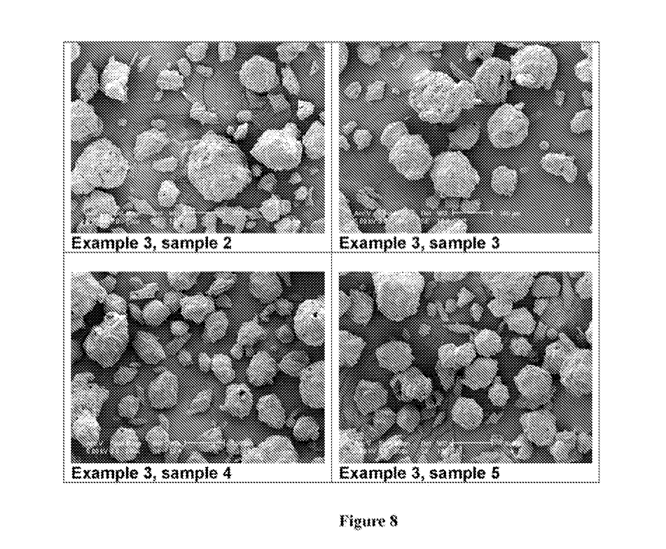

The excipient consists of microcrystalline cellulose at 89%, hydroxypropyl methyl cellulose at 2%, and crospovidone at 9%. The excipient was produced by a wet homogenization/spray drying granulation process. The apparatus used for the production of the excipient was a Co-current atomizer disc type with the disc RPM between 12000-25000 and the inlet temperatures of 180-250° C. After granulation a cyclone separation device was used to remove the fines. The production of the granular excipient begins with converting powdered MCC (which consists of rod like particles) into a slurry using deionized water in a mixing chamber to a concentration of 23.3%. In a separate container corspovidone was added to deionized water to form a 12.4% slurry. In another tank HPMC was added to deionized water to form a 7.3% slurry. One third of the MCCc slurry was transferred in a mixing tank and ⅖ of the crospovidone slurry was added to it under continuous stirring. This step was repeated until all the MCC and CPVD slurries were mixed together. The MCC/CPVD slurry was homogenized for 75 min. To the MCC/CPVD slurry was added the HPMC slurry and the final mixture was homogenized for 75 min. During the whole mixing process the homogenization is performed using a circulating shear pump and agitator. The resulting slurry mixture was then spray dried through a rotary nozzle at the motor frequency of 32.5 Hz in the presence of hot air at an outlet temperature of 106-109° C. This constitutes the granule formation step. The fines were removed in a cyclone and the final product was collected. The uniformity of product taken from several samplings is illustrated in FIG. 8.

The powder characteristics were determined as described in example 1.

TABLE 3

| Powder Characteristics | Value |
|---|---|
| 1. Compressibility | 16.5% |
| 2. D50 | 117 um |
| 3. Aerated bulk density | 0.27 g/cc |
| 4. Tapped bulk density | 0.34 g/cc |
| 5. LOD | 5.7% |

### EXAMPLE 4

### High Shear Wet Granulation of Microcrystalline Cellulose (89%)-HPMC (2%)-Crospovidone (9%):

133.5 g microcrystalline cellulose, 3.0 g Hydroxypropyl methylcellulose and 13.5 g crospovidone was placed in a 1 L stainless steel bowl. The bowl was attached to a GMX.01 vector micro high shear mixer/granulator (Vector Corporation). The dry mixture was mixed for 2 minutes at 870 rpm impeller speed and 1000 rpm chopper speed. 70 g of deionized water (“the liquid binder”) was added to the dry blend, drop by drop, using a peristaltic pump at a close rate of 16 rpm. During the liquid binder addition the impeller speed was 700 rpm and the chopper speed was 1500 rpm. The wet massing time was 60 seconds maintaining the same impeller and chopper speed as during the liquid addition. Following the granulation, the wet granular material was dried in a tray at 60° C. The resulting granular material (moisture content 2.4%) was screened through a 30 mesh sieve. The yield of the granular material that passed through 30 mesh screen was 116.7 g (79.3% referenced to dry starting materials and dry product). See FIG. 6.

### EXAMPLE 5

### Granule Friability Test for the Example 1 Excipient and the Material Obtained by High Shear Wet Granulation as per Example 4

75-100 g of granular material were loaded in a 4 L V-Blender and tumbled for 2 h. The granular material was collected and analyzed. An Air Jet Sieving instrument (Hosokawa Micron System) was used to determine the particle size distribution of the granular material before and after tumbling. A set of four sieves (270 mesh, 200 mesh, 100 mesh and 60 mesh) was used. The sieving time for each sieve was 60 sec, while the vacuum pressure was maintained at 12-14 in. H₂O. The sample size was 5 g.

TABLE 4

| | % Particles with diameter | % Particles with diameter |
|---|---|---|
| | less than 50 microns | less than 50 microns |
| Sample | before tumbling | after tumbling |
| Example 4 | 14 | 30 |
| Example 1 | 5 | 4 |

### EXAMPLE 6

### Comparison of Powder Characteristics for Example 1 and Example 3 Excipient and the Material Obtained by High Shear Wet Granulation as per Example 4

The powder characteristics of the granular materials were measured using a Powder Tester (Hosokawa Micron Corporation) Model PT-S. The Hosokawa Powder tester determines flowability of dry solids in accordance with the proven method of R. L. Carr. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm.

TABLE 5

| | Example 3 | | Example 4 | | Example 1 | |
|---|---|---|---|---|---|---|
| Property | Value | Index | Value | Index | Value | Index |
| Angle of repose (deg) | 30.9 | 22.0 | 37.9 | 18.0 | 34.9 | 20.0 |
| Aerated Bulk Density | 0.272 | | 0.299 | | 0.296 | |
| (g/cc) | | | | | | |
| Packed Bulk Density | 0.339 | | 0.389 | | 0.353 | |
| (g/cc) | | | | | | |
| Compressibility | 19.8 | 17.5 | 23.1 | 16.0 | 16.1% | 19.5 |
| Angle of Spatula | 31.6 | | 60.1 | | 44.6 | |
| Before Impact | | | | | | |
| Angle of Spatula After | 23.4 | | 42.5 | | 32.8 | |
| Impact | | | | | | |
| Angle of spatula (avg) | 27.5 | 24.0 | 51.3 | 16.0 | 38.7 | 19.5 |
| Uniformity | 2.9 | 23.0 | 2.9 | 23.0 | 2.1 | 23.0 |
| Total Flowability | | 86.5 | | 73.0 | | 82.0 |
| Index | | | | | | |

### EXAMPLE 7

### Comparison of Hardness vs. Compression Force Profiles for Placebo Tablets Prepared using Example 1 Excipient and the Material Obtained by High Shear Wet Granulation as per Example 4

Approximately 0.5 g tablets were pressed from the corresponding granular material at various compression forces using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in the table below are an average of three measurements.

TABLE 6

| | |
|---|---|
| Compression force | Hardness (kp) |

| (pound-force) | Example 4 | Example 1 |
|---|---|---|
| 3000 | 18.4 | 31.0 |
| 2000 | 12.9 | 22.2 |
| 1000 | 5.7 | 10.1 |

### EXAMPLE 8

### Comparison of Hausner Ratio and Can's Compressibility Index (A) of Microcrystalline Cellulose from Different Commercial Sources, Commercial Co-Processed Excipients Containing Microcrystalline Cellulose, and Example 1, 2 and 3 Excipients

Using the aerated and tapped bulk density, Carr's compressibility index and Hausner ratio can be calculated. A value of 20-21% or less for the Carr's compressibility index and a value below 1.25 for the Hausner ratio indicate a material with good flowability.

TABLE 7

| Excipient Brand Name | Hausner Ratio | Compressibility Index (%) |
|---|---|---|
| Emcocel 90 | 1.32 | 24.5 |
| Avicel PH 102 | 1.32 | 24.2 |
| Prosolv 90 | 1.23 | 18.9 |
| Example 4 | 1.30 | 23.1 |
| Example 2 | 1.25 | 19.7 |
| Example 1 | 1.19 | 16.1 |
| Example 3 | 1.22 | 16.5 |

Emcocel 90, Avicel PH 102 - brands of microcrystalline cellulose
Prosolv 90 - silicified microcrystalline cellulose

### EXAMPLE 9

### Disintegration Time vs. Hardness for Placebo Tablets of MCC Based Granular Excipient

Approximately 0.5 g tablets were pressed from the corresponding granular material at a compression force of 3000 lbs-f using a Carver manual press and a 13 mm die. The dwell time was 5 seconds. No lubricant was added. The disintegration experiments were performed with Distek Disintegration System 3100, using 900 mL deionoized water at 37° C.

TABLE 8

| Tablet | Hardness (kp) | Disintegration time (sec) |
|---|---|---|
| Example 1 | 31.0 | 56 |
| Example 2 | 30.3 | 150 |
| Example 3 | 26.3 | 42 |

### EXAMPLE 10

### Powder Properties of a Mixture of 5% Acetaminophen with Example 1 Excipient

7.9 g acetaminophen was blended with 150 g of Example 1 excipient in a 4 L V-blender for 1 h 30 min. The powder characteristics were measured using the same method mentioned in Example 6. The D50 value was calculated based on the data collected in a “particle size distribution” measurement similar to the one described in Example 5.

TABLE 9

| Powder Characteristics | Value |
|---|---|
| 1. Compressibility index | 20.7% |
| 2. D50 | 116 um |
| 3. Aerated bulk density | 0.29 g/cc |
| 4. Tapped bulk density | 0.36 g/cc |

### EXAMPLE 11

### Powder Properties of a Mixture of 30% Acetaminophen with Example 1 excipient

64.9 g acetaminophen was blended with 150 g of Example 1 excipient in a 4 L V-blender for 1 h 30 min. The powder characteristics were measured using the same method mentioned in Example 6. The D50 value was calculated based on the data collected in a “particle size distribution” measurement similar to the one described in Example 5.

TABLE 10

| Powder Characteristics | Value |
|---|---|
| 1. Compressibility index | 32.9% |
| 2. D50 | 117 um |
| 3. Aerated bulk density | 0.28 g/cc |
| 4. Tapped bulk density | 0.42 g/cc |

### EXAMPLE 12

### Powder Properties of a Mixture of 30% Ibuprofen with Example 1 Excipient

64.3 g ibuprofen was blended with 150 g of Example 1 excipient in a 4 L V-blender for 1 h 30 min. The powder characteristics were measured using the same method mentioned in Example 6. The D50 value was calculated based on the data collected in a “particle size distribution” measurement similar to the one described in Example 5.

TABLE 11

| Powder Characteristics | Value |
|---|---|
| 1. Compressibility index | 27.6% |
| 2. D50 | 105 um |
| 3. Aerated bulk density | 0.28 g/cc |
| 4. Tapped bulk density | 0.39 g/cc |

### EXAMPLE 13

### Preparation of 5% Acetaminophen Tablets using the Powder Blend Prepared According to Example 10

Approximately 0.5 g tablets were pressed from the corresponding granular material at various compression forces using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in the table below are an average of three measurements. The disintegration experiments were performed with a Distek Disintegration System 3100, using 900 mL deionoized water at 37° C.

TABLE 12

| Compression Force | Hardness | Disintegration |
|---|---|---|
| (pound-force) | (kp) | in Water |
| 4000 | 33.2 | 90 sec |
| 3000 | 28.3 | 52 sec |
| 2000 | 21.8 | 15 sec |

### EXAMPLE 14

### Preparation of 30% Acetaminophen Tablets Using the Powder Blend Prepared According to Example 11

Approximately 0.5 g tablets were pressed from the corresponding granular material at various compression forces using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in the table are an average of three measurements. The disintegration experiments were performed with a Distek Disintegration System 3100, using 900 mL deionized water at 37° C.

TABLE 13

| Compression Force | Hardness | Disintegration |
|---|---|---|
| (pound-force) | (kp) | in Water |
| 4000 | 17.4 | 18 sec |
| 3000 | 13.0 | 19 sec |
| 2000 | 8.8 | 16 sec |

### EXAMPLE 15

### Preparation of 50% Naproxen Sodium/Example 3

80 g naproxen sodium was blended with 80 g example 3 excipient and 800 mg (0.5%) amorphous silica (glidant) in a 4 L V-blender for 1 h 30 min. Approximately 0.5 g tablets were pressed from the corresponding granular material at various compression forces using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in the table below are an average of three measurements. The disintegration experiments were performed with a Distek Disintegration System 3100, using 900 mL deionoized water at 37 degrees Celsius.

TABLE 14
Hardness vs. Compression Force Profiles for Tablets
obtained from 50% Naproxen Sodium/Example 3 excipient

| Compression Force | Hardness |
|---|---|
| (lbs-force) | (kp) |
| 4000 | 16.8 |
| 3000 | 14.3 |
| 2000 | 11.8 |

TABLE 15
Disintegration time for Tablets obtained from 50%
Naproxen Sodium/Example 3 excipient

| Tablet Composition (hardness) | Disintegration time | |
|---|---|---|
| 50% Na Naproxen/Example 3 (16.8 kp) | 11 | min |
| 50% Na Naproxen/Example 3 (14.3 kp) | 10 in 20 | sec |

### EXAMPLE 16

Tabletability Study for a Blend of 62.5% Ibuprofen, Granular Excipient as per Example 1, Silica and Magnesium Stearate:

Ibuprofen, granular excipient as per Example 1 and Silica (see Table 16) were blended in a V-blender for 15 min at 20 rpm. The mixture was passed through a 30 mesh sieve and blended in a V-blender with Mg Stearate for 2 min at 20 rpm. The resulted blend was transferred to a 10 station rotary tableting machine (Mini Press II, Globe Pharma). Tablets were pressed using 10 mm dies and a force feeder operated at 10% power. Table 17 lists the tableting parameters used in the study.

TABLE 16

| Ingredient | | Amount (g) | % |
|---|---|---|---|
| 1 | Ibuprofen (Albemarle 20 um) | 1250 | 62.5 |
| 2 | Excipient as per Example 1 | 730 | 36.5 |
| 3 | Silica (RₓCipients ® GL100) | 10.0 | 0.5 |
| 4 | Mg Stearate (MBI) | 10.0 | 0.5 |
| | Total | 2000 | 100 |

TABLE 17

| | | | Compression | | Ejection | |
|---|---|---|---|---|---|---|
| Batch | % Motor | | force (lbs) | | (lbs) | |
| Name | Power | RPM | Average | % RSD | Average | % RSD |
| A | 30 | 10.4 | 3323.0 | 2.85 | 48.1 | 15.81 |
| B | 40 | 13.8 | 3223.4 | 3.58 | 49.6 | 10.33 |
| C | 50 | 17.6 | 2907.4 | 4.49 | 34.3 | 11.31 |
| D | 60 | 21.6 | 2798.9 | 5.16 | 31.0 | 13.24 |

### EXAMPLE 17

Characterization of the Ibuprofen Tablets as per Example 16:

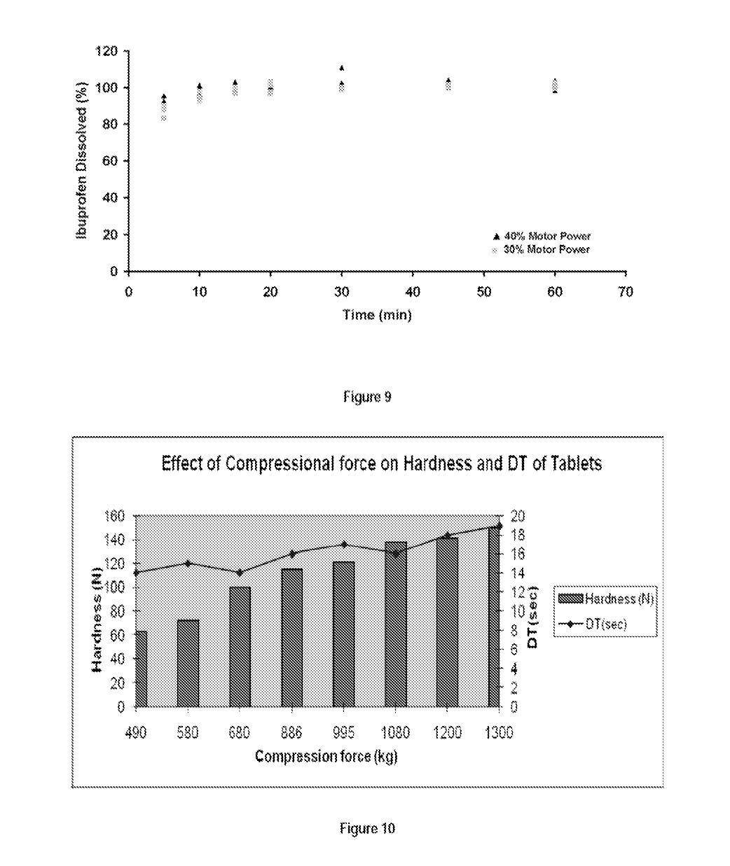

The ibuprofen tablets prepared as per Example 16 were characterized for tablet weight (Table 18), tablet thickness (Table 19), tablet hardness (Table 20), tablet friability (Table 21), tablet disintegration (Table 22) and Ibuprofen dissolution (FIG. 9).

The hardness and disintegration of the tablets were measured as described in Example 15. The tablet friability test was performed according to the USP recommendations for friability determination of compressed, uncoated tablets (see USP chapter <1216>) using a Varian Friabilator. The dissolution experiment was conducted according to the USP monograph for Ibuprofen tablets.

TABLE 18
Tablet weight (mg) for Ibuprofen tablets prepared
according to Example 16

| | Nr. tablets | | | | | |
|---|---|---|---|---|---|---|
| | for | | | | | |
| Batch name | statistics | MIN | MAX | Average | STDEV | % RSD |
| A | 25 | 321 | 339 | 329 | 3.68 | 1.12 |
| B | 25 | 314 | 327 | 321 | 3.65 | 1.14 |
| C | 25 | 297 | 319 | 307 | 5.52 | 1.80 |
| D | 25 | 300 | 322 | 309 | 6.79 | 2.20 |

TABLE 19
Tablet thickness (mm) for Ibuprofen tablets prepared according
to Example 16

| | Nr. tablets | | | | | |
|---|---|---|---|---|---|---|
| | for | | | | | |
| Batch name | statistics | MIN | MAX | Average | STDEV | % RSD |
| A | 25 | 4.64 | 4.76 | 4.72 | 0.034 | 0.72 |
| B | 25 | 4.53 | 4.71 | 4.63 | 0.053 | 1.15 |
| C | 25 | 4.46 | 4.62 | 4.53 | 0.047 | 1.03 |
| D | 25 | 4.43 | 4.67 | 4.54 | 0.070 | 1.55 |

TABLE 20
Tablet hardness (kp) for Ibuprofen tablets prepared according
to Example 16

| | Nr. tablets | | | | | |
|---|---|---|---|---|---|---|
| | for | | | | | |
| Batch name | statistics | MIN | MAX | Average | STDEV | % RSD |
| A | 25 | 8.3 | 12.1 | 10.4 | 1.03 | 9.93 |
| B | 25 | 7.8 | 10.8 | 9.5 | 0.88 | 9.26 |
| C | 25 | 5.2 | 8.5 | 7.3 | 0.91 | 12.34 |
| D | 25 | 4.9 | 8.1 | 6.4 | 0.73 | 11.44 |

TABLE 21
Tablet friability for Ibuprofen tablets
prepared according to Example 16

| | Weight before | Weight after | | |
|---|---|---|---|---|
| | tumbling | tumbling | Weight loss | Weight loss |
| Batch name | (g) | (g) | (g) | (%) |
| A | 6.602 | 6.583 | 0.019 | 0.29 |
| B | 6.801 | 6.787 | 0.014 | 0.21 |
| D | 6.773 | 6.748 | 0.025 | 0.37 |

TABLE 22
Tablet disintegration time (sec) for Ibuprofen
tablets prepared according to Example 16

| | Disintegration |
|---|---|
| Batch name | time (sec)* |
| A | 35 |
| B | 40 |
| C | 29 |
| D | 23 |

*Average for 4 tablets

### EXAMPLE 18

Tabletability of a Mixture of Excipient as per Example 1 and Mg Stearate using Various Tablet Weights, Punches and Embossing:

The Excipient as per Example 1 was passed through a 40 mesh sieve and Mg Stearate was passed through a 60 mesh sieve before mixing them with each other in a drum blender at a speed of 20 rpm for 2 min. Two batches were prepared according to Table 23. The lubricated blend of batch I was subdivided in 4 parts and the lubricated batch II was subdivided in two parts and taken for compression on a 16 station compression machine. The compression parameters are listed in Table 24. The effect of punch and embossing variation is given in Table 25.

TABLE 23

Batch No.

| | |
|---|---|
| Batch I | Batch II |

| | |
|---|---|
| Ingredients | mg/tablet |

| | | |
|---|---|---|
| Excipient as per example 1 | 498.75 | 997.5 |
| Magnesium Stearate | 1.25 | 2.50 |
| Tablet weight (mg) | 500 | 1000 |

TABLE 24

| | | | | Weight | Hardness | Disintegration Time |
|---|---|---|---|---|---|---|
| Batch | Sub-batch | Punch | Embossing | (mg/tablet) | (kp) | (sec) |
| I | IA | Circular, | “EM 400” with | 500 | 7.3 | 17 |
| | IB | 11 mm | Break line | | 12.8 | 18 |
| | | Beveled | On upper | | | |
| | | edges | Punch | | | |
| | IC | Oval, | “IRH” - upper | 500 | 7.1 | 17 |
| | ID | 15.5 × 8.0 mm | Punch | | 12.8 | 17 |
| | | | “200” - lower | | | |
| | | | punch | | | |
| II | IIA | Circular, | No embossing | 1000 | 7.1 | 19 |
| | IIB | 11 mm | “EM 400” - | | 12.3 | 19 |
| | | | break line | | | |

TABLE 25

| | | Sticking/picking | Weight | Effect on | Effect on |
|---|---|---|---|---|---|
| Batch | Sub-batch | to the punches | variation | hardness | embossing |
| I | A | No | No | No | No |
| | B | No | No | No | No |
| | C | No | No | No | No |
| | D | No | No | No | No |
| II | A | No | No | No | No |
| | B | No | No | No | No |

### EXAMPLE 19

Coating of Tablets Prepared from the Example 1 Excipient:

345 g tablets pressed utilizing Example 1 excipient were coated with 62.5 g of 18% orange OPADRY® (Coloreon) suspension in water. The tablet coater used was FREUND Model HCT-30 HI-COATER. The pump rate was set to 14 g/min. The inlet air temperature was 80 C, the outlet air temperature was 34-36 C. the pan rotation was 20 rpm and the air nozzle pressure was 16 psi.

The resulted coated tablets were defect free and uniformly coated.

### EXAMPLE 20

Properties of Blends Consisting of Example 1 Excipient and a Filler:

Blends of Example 1 excipient and a filler in 4:1, 2:1 and 1:1 ratio (by weight) were prepared by blending the components in a V-blender for 30 min-1 h. The fillers used in this study were: microcrystalline cellulose, spray dried lactose and dibasic calcium phosphate. The resulted blends were characterized for particle size distribution, aerated bulk density and tapped bulk density using the same methods as described in Example 1. Tabletability was done using a Carver manual press and a 13 mm die with no lubricant added. The results are presented in Table 26, 27 and 28, respectively.

TABLE 26
Characterization of Example 1 excipient - Microcrystalline Cellulose Blends

| Example 1 | d₁₀ | | Aerated | Tapped | | Hardness | | |
|---|---|---|---|---|---|---|---|---|
| excipient:MCC | d₅₀ | % retained | Bulk | Bulk | Compressib | (kp) | | |
| (by | d₉₀ | on 200 | Density | Density | Index | 2000 lbs- | 3000 lbs- | Disintegration |
| weight)^{a} | (um) | mesh | (g/cc) | (g/cc) | (%) | force | force | Time (sec) |
| 1:0 | 59.6 | 75.55 | 0.296 | 0.353 | 16.1 | 22.2 | 31.0 | 25 |
| | 113.3 | | | | | | | (for 22.2 |
| | 170.8 | | | | | | | kp) |
| 4:1 | 71.0 | 79.30 | 0.317 | 0.371 | 14.6 | 21.5 | 26.7 | 22 |
| | 123.0 | | | | | | | (for 21.5 |
| | 175.4 | | | | | | | kp) |
| 2:1 | 60.2 | 79.17 | 0.302 | 0.368 | 17.9 | 22.1 | 27.1 | 11 |
| | 119.0 | | | | | | | (for 22.1 |
| | 186.1 | | | | | | | kp) |
| 1:1 | 54.6 | 78.74 | 0.308 | 0.367 | 16.1 | 20.8 | 27.1 | 12 |
| | 118.0 | | | | | | | (for 20.8 |
| | 192.4 | | | | | | | kp) |

^{a}The MCC used in the study was MCC 102 RanQ and had the following properties: d₁₀ = 37.4 um; d₅₀ = 94.6 um; d₉₀ = 192.6 um; aerated bulk density = 0.298 g/cc; tapped bulk density = 0.403 g/cc; % Compressibility Index = 26.1.

The Table 27 Characterization of Example 1 excipient - Spray dried lactose Blends

| Example 1 | D₁₀ | | Aerated | Tapped | | | | |
|---|---|---|---|---|---|---|---|---|
| excipient:Lactose | d₅₀ | % retained | Bulk | Bulk | | Hardness | | |
| (by | d₉₀ | on 200 | Density | Density | Compressib | 2000 lbs- | 3000 lbs- | Disintegration |
| weight)^{a} | (um) | mesh | (g/cc) | (g/cc) | Index | force | force | Time (sec) |
| 1:0 | 59.6 | 75.55 | 0.296 | 0.353 | 16.1 | 22.2 | 31.0 | 25 |
| | 113.3 | | | | | | | (for 22.2 |
| | 170.8 | | | | | | | kp) |
| 4:1 | 58.2 | 82.35 | 0.352 | 0.400 | 12.0 | 15.6 | 20.5 | 25 |
| | 116.5 | | | | | | | (for 20.5 |
| | 181.2 | | | | | | | kp) |
| 2:1 | 64.9 | 86.15 | 0.369 | 0.435 | 15.2 | 11.5 | 16.2 | 22 |
| | 127.4 | | | | | | | (for 16.2 |
| | 195.7 | | | | | | | kp) |
| 1:1 | 56.9 | 81.15 | 0.416 | 0.470 | 11.5 | 11.1 | 15.2 | 16 |
| | 117.3 | | | | | | | (for 15.2 |
| | 186.1 | | | | | | | kp) |

^{a}The Lactose used in the study was spray dried Supertab—New Zeeland and has the following properties: d₁₀ = 54.25 um; d₅₀ = 118.65 um; d₉₀ = 195.4 um; aerated bulk density = 0.616 g/cc; tapped bulk density = 0.762 g/cc; % Compressibility Index = 19.2

TABLE 28
Characterization of Example 1 excipient - Dibasic Calcium Phosphate (DCP)

| Example 1 | D₁₀ | | Aerated | Tapped | | | | |
|---|---|---|---|---|---|---|---|---|
| excipient:DCP | d₅₀ | % retained | Bulk | Bulk | | Hardness | | |
| (by | d₉₀ | on 200 | Density | Density | Compressib | 2000 lbs- | 3000 lbs- | Disintegration |
| weight)^{a} | (um) | mesh | (g/cc) | (g/cc) | Index | force | force | Time |
| 1:0 | 59.6 | 75.55 | 0.296 | 0.353 | 16.1 | 22.2 | 31.0 | 25 |
| | 113.3 | | | | | | | (for 22.2 |
| | 170.8 | | | | | | | kp) |
| 4:1 | 77.311 | 91.55 | 0.360 | 0.422 | 15.7 | 18.7 | 22.1 | 51 |
| | 45.1 | | | | | | | (for 22.1 |
| | 216.6 | | | | | | | kp) |
| 2:1 | 62.9 | 85.9 | 0.399 | 0.461 | 13.4 | 15.9 | 20.4 | 31 |
| | 137.6 | | | | | | | (for 20.4 |
| | 226.4 | | | | | | | kp) |
| 1:1 | 60.2 | 85.1 | 0.465 | 0.540 | 15.2 | 12.0 | 15.0 | 18 |
| | 144.7 | | | | | | | (for 15.0 |
| | 253.1 | | | | | | | kp) |

^{a}The DCP used in the study was A-TAB (Rhodia) and has the following properties: d₁₀ = 60.7 um; d₅₀ = 188.0 um; d₉₀ = 389.0 um; aerated bulk density = 0.753 g/cc; tapped bulk density = 0.861 g/cc; % Compressibility Index = 12.5.

### EXAMPLE 21

Tabletability Study for a Formulation of Atorvastatin Calcium that uses Example 1 Excipient:

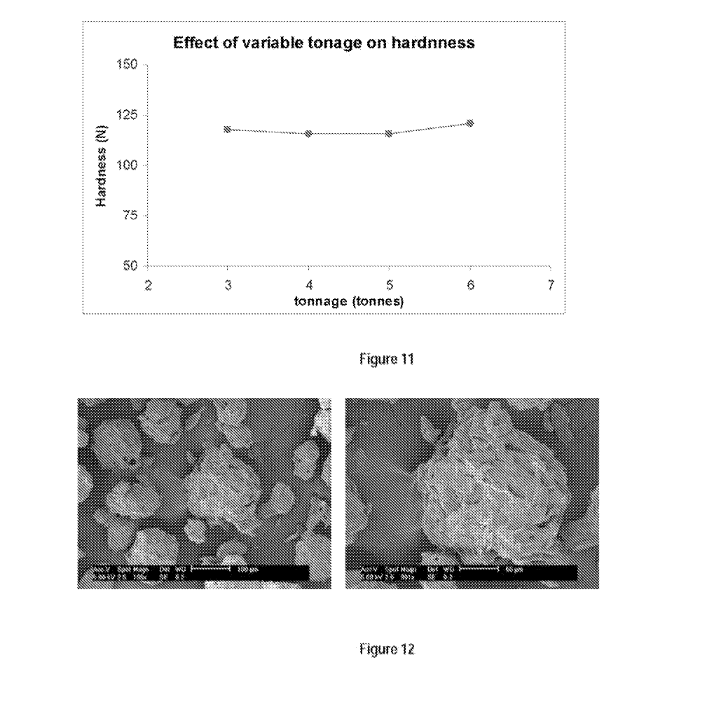

A 3000 tablet batch size of a formulation (Table 29) of atorvastatin calcium (a crystalline form) was prepared using a 16 station compression machine. The compression parameters are listed in Table 30. The effect of varying compression pressure on tablet hardness and tablet disintegration time was studied (FIG. 10). The effect of varying tonnage on hardness was also studied (FIG. 11).

TABLE 29

| Ingredients | mg/tablet |
|---|---|
| Atorvastatin Calcium | 80.0 |
| Example 1 excipient | 478.0 |
| CaCO₃ | 240.0 |
| Magnesium Stearate | 2.0 |
| Tablet weight | 800.0 |
| Batch size | 3000 tablets |

TABLE 30

| | | | Tablet | | Disintegration |
|---|---|---|---|---|---|
| | | Em- | weight | Hardness | time |
| Batch | Punch | bossing | (mg/tablet) | (kp) | (sec) |
| 1 | Kite | None | 767-817 | 24.0 | 17 |
| 2 | shape, | | 784-807 | 14.2 | 18 |
| 3 | 18 × 11 | | 779-790 | 6.9 | 15 |

### EXAMPLE 22

Preparation of Microcrystalline Cellulose—2% Hydroxypropyl Methylcellulose Excipient According to the Present Invention:

The alternate improved excipient consists of 98% microcrystalline cellulose and 2% hydroxypropyl ruethyleellulose. The excipient was produced by a wet homogenization/spray dry granulation process. The apparatus used for the production of the excipient was a Co-current atomizer disc type with the disc RPM between 12000 and 25000 and the inlet temperature of 180-250° C. Powdered MCC was converted into a slurry with deionized water to give a concentration of 23.58% w/w. in a separate slurry tank, the HPMC was mixed with deionized water, stirred and circulated for 60-70 min to give a concentration of 16.11% w/w. The prepared HPMC slurry was added to the MCC slurry. The HPMC slurry tank was washed with 5 L of water and the washings were added to the MCC/HPMC slurry. The resulted mixture was stirred, circulated and homogenized into a uniform slurry of 23.09% concentration for 85 min using a circulating shear pump and an agitator to keep solid suspended. The slurry mixture was then spray dried through a rotary nozzle at a motor frequency of 35 Hz in the presence of hot air at an outlet temperature of 102-109° C. This constitutes the granule formation step. The fines were removed in a cyclone and the final product was collected to give the alternate improved excipient. SEM micrographs of the excipient of Example 22 are seen in FIG. 12. Unless otherwise noted, all SEM rnicrographs herein were recorded using a FEI XL30 ESEM (environmental scanning electron microscope), voltage 5 kV, spot size 3, SE detector. The samples were sputtered with Iridium before SEM analysis (sputtering time 40 sec).

The compressibility, aerated bulk density and tapped bulk density of the granular material were measured using a Powder Tester (Hosokawa Micron Corporation) Model PT-S (Table 31). A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm. D50 value was calculated based on the data collected in a “particle size distribution” measurement. An Air Jet Sieving instrument (Hosokawa Micron System) was used to determine the particle size distribution of the granular material. A set of four sieves (270 mesh, 200 mesh, 100 mesh and 60 mesh) was used. The sieving time for each sieve was 60 sec, while the vacuum pressure was maintained at 10-12 in. H₂O. The sample size was 5 g.

The “loss on drying” (LOD) value was determined using a Mettler Toledo Infrared Dryer LP16. The set temperature was 120° C. and the analysis was stopped when constant weight was reached.

TABLE 31

| Powder Characteristic | Value |
|---|---|
| Angle of repose (°) | 31.3 |
| Aearted Bulk Density (g/cc) | 0.274 |
| Tapped Bulk Desnity (g/cc) | 0.346 |
| Compressibility (%) | 20.8 |
| Hausner ratio | 1.26 |
| D50 (um) | 109.5 |
| LOD (%) | 2.5 |

### EXAMPLE 23

Preparation of Microcrystalline Cellulose—5% Hydroxypropyl Methylcellulose Excipient According to the Present Invention:

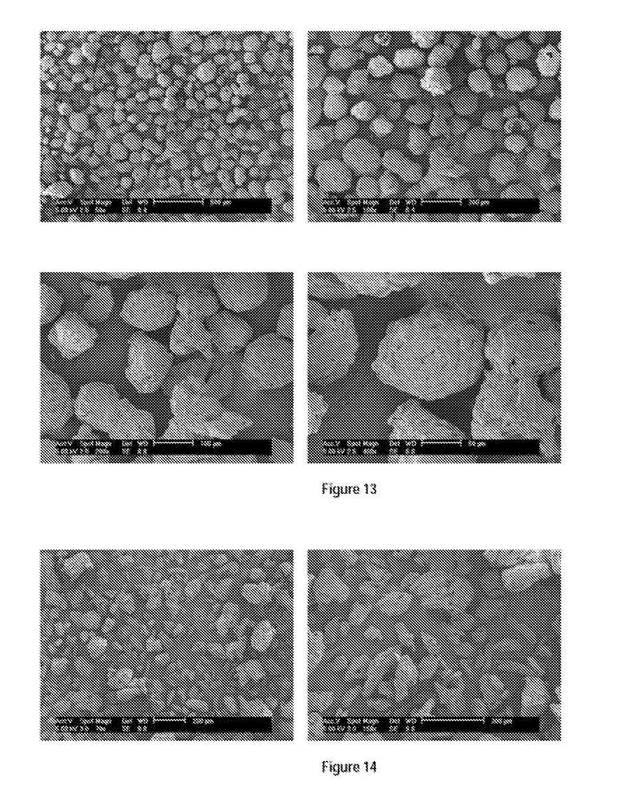

This embodiment of the alternate improved excipient consists of 95% microcrystalline cellulose and 5% hydroxypropyl methylcellulose. The excipient as produced by a wet homogenization/spray dry granulation process. The apparatus used for the production of the excipient was a Co-current atomizer disc type with the disc RPM between 12000 and 25000 and the inlet temperature of 180-250° C. Powdered MCC was converted into a slurry with deionized water to give a concentration of 23.0% w/w. In a separate slurry tank, the HPMC was mixed with deionized water, stirred and circulated for 60-70 min to give a concentration of 17.20% w/w. The prepared HPMC slurry was added to the MCC slurry. The HPMC slurry tank was washed with 5 L of water and the washings were added to the MCC slurry. The resulted mixture together with additional deionized water was stirred, circulated and homogenized into a uniform slurry of 22.44% concentration for 60 min using a circulating shear pump and an agitator to keep solid suspended. The slurry mixture was then spray dried through a rotary nozzle at a motor frequency of 35 Hz in the presence of hot air at an outlet temperature of 104-110° C. This constitutes the granule formation step. The fines were removed in a cyclone and the final product was collected to give the new improved excipient. SEM micrographs of the excipient of Example 23 are seen in FIG. 13.

The powder characteristics (Table 32) were determined as described in example 22.

TABLE 32

| Powder Characteristic | Value |
|---|---|
| Angle of repose (°) | 31.5 |
| Aearted Bulk Density (g/cc) | 0.236 |
| Tapped Bulk Desnity (g/cc) | 0.298 |
| Compressibility (%) | 20.8 |
| Hausner ratio | 1.26 |
| D50 (um) | 135.49 |
| LOD (%) | 2.1 |

### EXAMPLE 24

High Shear Wet Granulation of Microcrystalline Cellulose (98%)-HPMC (2%):

147 g microcrystalline ceIiuose and 3.0 g Hydroxypropyl methylcellulose were placed in a 1 L stainless steel bowl. The bowl was attached to a GMX.01 vector micro high shear mixer/granulator (Vector Corporation). The dry mixture was mixed for 2 minutes at 870 rpm impeller speed and 1000 rpm chopper speed. 70 g of deionized water (“the liquid binder”) was added to the dry blend, drop by drop, using a peristaltic pump at a dose rate of 16 rpm. During the liquid binder addition the impeller speed was 700 rpm and the chopper speed was 1500 rpm. The wet massing time was 60 seconds maintaining the same impeller and chopper speed as during the liquid addition. Following the granulation, the wet granular material was dried in a tray at 60° C. The resulted granular material (moisture content 2.00%) was screened through a 30 mesh sieve. The yield of the granular material that passed through 30 mesh screen was 137.7 g (94.1% referenced to dry starting materials and dry product). SEM micrographs of the granular material of Example 24 are seen in FIG. 14.

### EXAMPLE 25

High Shear Wet Granulation of Microcrystalline Cellulose (95%)-HPMC (5%):

142.5 g microcrystalline cellulose and 7.5 g Hydroxypropyl methylcellulose were placed in a 1 L stainless steel bowl. The bowl was attached to a GMX.01 vector micro high shear mixer/granulator (Vector Corporation). The high shear wet granulation process was conducted as in Example 24. The resulted granular material (moisture content 2.95%) was screened through a 30 mesh sieve. The yield of the granular material that passed through 30 mesh screen was 113.15 g (76.5% referenced to dry starting materials and dry product).

### EXAMPLE 26

High Shear Wet Granulation of Microcrystalline Cellulose (90%)-HPMC (10%):

135.0 g microcrystalline cellulose and 15.0 g Hydroxypropyl methylcellulose were placed in a 1 L stainless steel bowl. The bowl was attached to a GMX.01 vector micro high shear mixer/granulator (Vector Corporation). The high shear wet granulation process was conducted as in Example 24 with the exception that the amount of water (“liquid binder”) added was 66 g. The resulted granular material (moisture content 4.5%) was screened through a 30 mesh sieve. The yield of the granular material that passed through 30 mesh screen was 79.95 g (53.1% referenced to dry starting materials and dry product).

### EXAMPLE 27

Powder blend of Microcrystalline Cellulose and Hydroxypropyl methylcellulose:

Predetermined amounts (see Table 33) of microcrystalline cellulose and Hydroxypropyl methylcellulose were blended hi a V-blender for an hour.

TABLE 33

| | Microcrystalline Cellulose | Hydroxypropyl methylcellulose |
|---|---|---|
| Example | (g) | (g) |
| 27a | 147 | 3 |
| 27b | 142.5 | 7.5 |
| 27c | 135 | 15 |

### EXAMPLE 28

Comparison of Powder Characteristics for Example 22, 23, 27a, 27b Excipients and Two Commercial Brands of Microcrystalline Cellulose (Avicel 102 and MCC 102 -RanQ):

The powder properties (Tables 34 and 35) of the materials prepared in Example 22, 23, 27a, 27b, Avicel 102 and MCC102-RanQ were measured using a Powder Tester (Hosokawa Micron Corporation) Model PT-S. The Hosokawa Powder tester determines flowability of dry solids in accordance with the proven method of R. L. Carr. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm.

TABLE 34
Powder Characteristics for materials prepared according to Examples 22, 23 and two
commercially available microcrystalline cellulose brands
(Avicel 102; MCC102-RanQ)

| | | | | | | | MCC 102- | |
|---|---|---|---|---|---|---|---|---|
| | Example 22 | | Example 23 | | Avicel 102 | | RanQ | |
| Property | Value | Index | Value | Index | Value | Index | Value | Index |
| Angle of repose | 31.3 | 22.0 | 31.5 | 21.0 | 37.1 | 18.0 | 37.1 | 18.0 |
| (deg) | | | | | | | | |
| Aerated Bulk | 0.274 | | 0.236 | | 0.345 | | 0.298 | |
| Density (g/cc) | | | | | | | | |
| Packed Bulk | 0.346 | | 0.298 | | 0.455 | | 0.403 | |
| Density (g/cc) | | | | | | | | |
| Compressibility | 20.8 | 17.0 | 20.8 | 17.0 | 24.2 | 16.0 | 26.1 | 14.5 |
| (%) | | | | | | | | |
| Angle of Spatula | 28.3 | | 26.8 | | 35.9 | | 36.8 | |
| Before Impact | | | | | | | | |
| Angle of Spatula | 22.6 | | 23.4 | | 32.5 | | 29.6 | |
| After Impact | | | | | | | | |
| Angle of spatula | 25.5 | 25.0 | 25.1 | 25.0 | 34.2 | 21.0 | 33.2 | 21.0 |
| (avg) | | | | | | | | |
| Uniformity | 2.3 | 23.0 | 2.0 | 23.0 | 3.4 | 23.0 | 2.8 | 23.0 |
| Total Flowability | | 87.0 | | 86.0 | | 78.0 | | 76.5 |
| Index | | | | | | | | |

TABLE 35
Powder Characteristics for MCC-HPMC powder blends
prepared according to Examples 27a and 27b

| | Example 27a | | Example 27b | |
|---|---|---|---|---|
| Property | Value | Index | Value | Index |
| Angle of repose (deg) | 36.5 | 18.0 | 35.6 | 19.5 |
| Aerated Bulk Density (g/cc) | 0.304 | | 0.313 | |
| Packed Bulk Density (g/cc) | 0.401 | | 0.407 | |
| Compressibility (%) | 17.8 | 18.0 | 23.1 | 16.0 |
| Angle of Spatula Before Impact | 34.7 | | 28.2 | |
| Angle of Spatula After Impact | 31.3 | | 24.0 | |
| Angle of spatula (avg) | 33.0 | 21.0 | 26.1 | 24.0 |
| Uniformity | 2.8 | 23.0 | 2.9 | 23.0 |
| Total Flowability Index | | 78.0 | | 82.5 |

### EXAMPLE 29

Comparison of Hausner Ratio and Carr's Compressibility Index (%) of Microcrystal Cellulose from Different Commercial Sources, and Example 22 and 23

Using the aerated and tapped bulk density, Carr's compressibility index and Hausner ratio can be calculated (Table 36). A value of 20-21% or less for the Can's compressibility index indicate a material with good flowability.

TABLE 36

| | Hausner | Compressibility Index |
|---|---|---|
| Excipient Brand Name | Ratio | (%) |
| Emcocel 90 | 1.32 | 24.5 |
| Avicel PH 102 | 1.32 | 24.2 |
| MCC 102 RanQ | 1.35 | 26.1 |
| Example 22 | 1.26 | 20.8 |
| Example 23 | 1.26 | 20.8 |

### EXAMPLE 30

Granule Friability Test for Example 23 and Example 25 Excipients

75-100 g of granular material was analyzed for particle size distribution and then was loaded in a 4 L V-Blender and tumbled for 2 h. The granular material was collected and analyzed again for particle size distribution (Table 37). An Air Jet Sieving instrument (Hosokawa Micron System) was used to determine the particle size distribution of the granular material before and after tumbling. A set of four sieves (270 mesh, 200 mesh, 100 mesh and 60 mesh) was used. The sieving time for each sieve was 60 see, while the vacuum pressure was maintained at 12-14 in. H₂O. The sample size was 5 g.

TABLE 37

| | % Particles with diameter | % Particles with diameter |
|---|---|---|
| | less than 50 microns | less than 50 microns |
| Sample | before tumbling | after tumbling |
| Example 22 | 2.10 | 2.83 |
| Example 25 | 1.68 | 5.87 |

### EXAMPLE 31

Comparison of Hardness vs. Compression Force for Placebo Tablets Prepared using Example 22, Example 23, Example 24 and Example 25 Excipient, Respectively (Table 38):

Approximately 0.5 g tablets were pressed from the corresponding excipient at various compression forces using a Carver manual press and a 13 min die. The dwell time was 5 seconds. No lubricant was added. The hardness of the tablets was measured using a Varian, Benchsaver™ Series, VK 200 Tablet Hardness Tester. The values recorded in the table bellow are an average of three measurements.

TABLE 38

| Compression | | | | |
|---|---|---|---|---|
| force | Hardness (kp) | | | |
| (pound-force) | Example 22 | Example 23 | Example 24 | Example 25 |
| 2000 | 15.3 | 17.0 | 12.2 | 8.5 |
| 3000 | 21.5 | 22.4 | 15.7 | 13.4 |

### EXAMPLE 32

High Shear Wet Granulation of Example 23 Excipient:

150 g excipient prepared as per Example 23 were placed in a 1 L stainless steel bowl. The bowl was attached to a GMX.01 vector micro high shear mixer/granulator (Vector Corporation). The high shear wet granulation process was conducted as in Example 24. The resulted granular material (moisture content 3%) was screened through a 30 mesh sieve.

### EXAMPLE 33

High Shear Wet Granulation of Microcrystalline Cellulose:

150 g microcrystalline cellulose MCC102RanQ were placed in a 1 L stainless steel bowl. The bowl was attached to a GMX.01 vector micro high shear mixer/granulator (Vector Corporation). The high shear wet granulation process was conducted as in Example 24. The resulted granular material (moisture content 3%) was screened through a 30 mesh sieve.

### EXAMPLE 34

Comparison of Hausner Ratio and Carr's Compressibility Index (%) of the Granular Materials Prepared as per Example 32 and Example 33, Respectively:

Using the aerated and tapped bulk densities, Carr's compressibility index and Hausner ratio can be calculated (Table 39).

TABLE 39

| Granular material | Example 32 | Example 33 |
|---|---|---|
| Aerated bulk density (g/cc) | 0.321 | 0.372 |
| Tapped bulk density (g/cc) | 0.373 | 0.458 |
| Compressibility Index (%) | 13.9 | 18.8 |
| Hausner ratio | 1.16 | 1.23 |

### EXAMPLE 35

Tablet hardness for placebo tablets of the granular materials prepared as per Example 32 and Example 33. respectively and their comparison with tablet hardness for placebo tablets of MCC 102 RanQ and excipient prepared as per example 23:

Approximately 0.5 g tablets were pressed from the corresponding excipient at 3000 lbs-force compression force using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in Table 40 are an average of four measurements.

TABLE 40

| | | | MCC 102 | |
|---|---|---|---|---|
| Granular material | Example 23 | Example 32 | RanQ | Example 33 |
| Tablet | 22.4 | 21.25 | 32.13 | 23.57 |
| Hardness (kp) | | | | |

### EXAMPLE 36

Powder Characteristics of a Mixture Consisting of Excipient Prepared as Per Example 22 and 9% Disintegrant:

455.0 g of excipient from example 22 and 45.0 g crospovidone (disintegrant) were blended in a V-blender for 30 mm. powder characteristics were determined as described in 22 and are presented in Table 41.

TABLE 41

| Powder Characteristic | Value |
|---|---|
| Angle of repose (°) | 38.9 |
| Aearted Bulk Density (g/cc) | 0.250 |
| Tapped Bulk Desnity (g/cc) | 0.332 |
| Compressibility (%) | 24.7 |
| Hausner ratio | 1.328 |
| D50 (um) | 105.37 |

### EXAMPLE 37

Tableting Study of the Excipient Mixture Prepared as per Example 36:

250.0 g of the excipient mixture prepared as per example 36 and 0.625 g Magnesium Stearate (lubricant) were blended in a V-blender for 2 min. Placebo tablets were pressed on a 10 stations rotary tablet press, Mini Press II, Globe Pharma using 10 mm dies. The tableting machine operated at a 40% motor power (13.7 rpm), The compression force was 1300 lbs and the ejection force was 12.9 lbs. The tablet characteristics are presented in Table 42.

TABLE 42

| Tablet characteristic | Average | % RSD |
|---|---|---|
| Tablet weight (mg) | 268* | 1.32 |
| Tablet thickness (mm) | 4.32* | 0.44 |
| Tablet hardness (kp) | 12.4* | 6.20 |
| Tablet disintegration (sec) | 24** | 15.07 |

*average over 25 tablets randomly selected from the batch
**average over 8 tablets randomly selected from the batch

### EXAMPLE 38

Powder Characteristics of a Mixture Prepared from Ibuprofen (63%), the Blend Prepared as per Example 36 and Silica:

70.0 g Ibuprofen (20 um), 40.57 g blend prepared as per example 36 and 0.54 g silica were blended for 30 min in a V-blender. The powder characteristics were determined as described in example 22 and are presented in Table 43.

TABLE 43

| Powder Characteristic | Value |
|---|---|
| Angle of repose (°) | 37.2 |
| Aearted Bulk Density (g/cc) | 0.379 |
| Tapped Bulk Desnity (g/cc) | 0.546 |
| Compressibility (%) | 30.6 |
| Hausner ratio | 1.44 |
| D50 (um) | 35.67 |

### EXAMPLE 39

Tableting Study of the Blend Prepared as Per Example 38:

100.0 g of the mixture prepared as per example 38 and 1.0 g Magnesium Stearate (lubricant) were blended in a V-blender for 2 min. Ibuprofen tablets were pressed on a 10 stations rotary tablet press, Mini Press II, Globe Pharma using 10 mm dies. The tableting machine operated at 7.0 rpm. The compression force was 2600 lbs and the ejection force was 53 lbs. The tablet characteristics are presented in Table 44.

TABLE 44

| Tablet characteristic | Average | % RSD |
|---|---|---|
| Tablet weight (mg) | 305* | 2.26 |
| Tablet thickness (mm) | 4.40* | 1.18 |
| Tablet hardness (kp) | 10.0* | 8.83 |
| Tablet disintegration (sec) | 45** | 15.84 |

*average over 25 tablets randomly selected from the batch
**average over 4 tablets randomly selected from the batch

Having described the invention in detail, those skilled in the art will appreciate that modifications may be made of the invention without departing from its' spirit and scope. Therefore, it is not intended that the scope of the invention be limited to the specific embodiments described. Rather, it is intended that the appended claims and their equivalents determine the scope of the invention.

Unless otherwise noted, all percentages are weight/weight percentages.

1. A composition comprising:
about 90% to about 99% microcrystalline cellulose; and
about 1% to about 10% at least one binder;
wherein the microcrystalline cellulose and binder are indistinguishable when viewed with a SEM, thereby forming substantially homogeneous-particles.

2. The composition of claim 1 wherein the composition includes:
about 95% to about 99% microcrystalline cellulose; and
about 1% to about 5% at least one binder.

3. The composition of claim 1 wherein the composition includes:
about 97% to about 99% microcrystalline cellulose; and
about 1% to about 3% at least one binder.

4. The composition of claim 1 wherein the binder includes hydroxypropyl methylcellulose.

5. The composition of claim 1 wherein the excipient is formed by homogenizing/spray dry granulating an aqueous slurry comprised of the microcrystalline cellulose and binder.

6. The composition of claim 1 wherein the aerated bulk density is 0.2-0.3 g/cc.

7. A method or making an excipient comprising:
mixing a microcrystalline cellulose (MCC) slurry with a cross-linked polyvinylpyrrolidone slurry to form a MCC/cross-linked polyvinylpyrrolidone slurry;
mixing a binder comprising hydroxypropyl methylcellulose in water to form a viscous hydroxypropyl methylcellulose slurry;
homogenizing the hydroxypropyl methylcellulose slurry with the MCC/cross-linked polyvinylpyrrolidone slurry to form a homogenized slurry; and
spray dry granulating the homogenized slurry through a rotary nozzle at an RPM of between 12,000 and 25,000 to form substantially homogeneous and substantially spherical particles of excipient wherein the microcrystalline cellulose and hydroxypropyl methylcellulose are indistinguishable when viewed with a SEM, and wherein said excipient is a complete and universal directly compressible excipient with a flowability index over 80.

8. (canceled)

9. (canceled)

10. (canceled)

11. (canceled)

12. (canceled)

13. (canceled)

14. (canceled)

15. (canceled)

16. pharmaceutical tablet comprising:
at least one active pharmaceutical ingredient;
a disintegrant; and
an excipient of substantially homogeneous particles including:
a) microcrystalline cellulose; and
b) at least one binder.

17. The tablet of claim 16 wherein the excipient includes:
about 90% to about 99% microcrystalline cellulose; and
about 1% to about 10% at least one binder.

18. The tablet of claim 16 wherein the excipient includes:
about 95% to about 99% microcrystalline cellulose; and
about 1% to about 5% at least one binder.

19. The tablet of claim 16 wherein the excipient includes:
about 97% to about 99% microcrystalline cellulose; and
about 1% to about 3% at least one binder.

20. The tablet of claim 16 wherein the binder includes hydroxypropyl methylcellulose.

21. A method of making a pharmaceutical tablet comprising:
mixing at least one active pharmaceutical ingredient with a disintegrant and an excipient of substantially homogeneous particles including:
a) microcrystalline cellulose; and
b) at least one binder; and
compressing the mixture to form a tablet.

22. The method of claim 21 wherein the excipient includes:
about 90% to about 99% microcrystalline cellulose; and
about 1% to about 10% at least one binder.

23. The method of claim 21 wherein the excipient includes:
about 95% to about 99% microcrystalline cellulose; and
about 1% to about 5% at least one binder.

24. The method of claim 21 wherein the excipient includes:
about 97% to about 99% microcrystalline cellulose; and
about 1% to about 3% at least one binder.

25. The method of claim 21 wherein the binder includes hydroxypropyl methylcellulose.

26. A composition comprising:
about 75% to about 98% microcrystalline cellulose;
about 1% to about 10% at least one binder; and
about 1% to about 20% at least one disintegrant;
wherein the microcrystalline cellulose, binder and disintegrant are indistinguishable when viewed with a SEM, thereby forming substantially homogeneous, substantially spherical particles.

27. The composition of claim 26 wherein the composition includes:
about 80% to about 90% microcrystalline cellulose;
about 2% to about 8% at least one binder; and
about 3% to about 12% at least one disintegrant.

28. The composition of claim 26 wherein the composition includes:
about 85% to about 93% microcrystalline cellulose;
about 2% to about 5% at least one binder; and
about 10% at least one disintegrant.

29. The composition of claim 26 wherein the binder includes hydroxypropyl methylcellulose and the disintegrant includes cross-linked polyvinylpyrrolidone.

30. The composition of claim 26 wherein the excipient is formed by spraying an aqueous slurry comprised of the microcrystalline cellulose, binder and disintegrant.

31. A method of making an excipient comprising:
mixing a MCC slurry with a disintegrant slurry to form a MCC/disintegrant slurry; mixing a binder in water to form a viscous binder slurry;
homogenizing the binder slurry with the MCC/disintegrant slurry to form a homogenized slurry; and
spray dry granulating the homogenized slurry to form substantially homogeneous, substantially spherical particles of excipient.

32. The method of claim 31 wherein:
about 75% to about 98% microcrystalline cellulose;
about 1% to about 10% at least one binder; and
about 1% to about 20% at least one disintegrant.

33. The method of claim 31 comprising:
about 80% to about 90% microcrystalline cellulose;
about 2% to about 8% at least one binder; and
about 3% to about 12% at least one disintegrant.

34. The method of claim 31 comprising:
about 85% to about 93% microcrystalline cellulose;
about 2% to about 5% at least one binder; and
about 10% at least one disintegrant.

35. The method of claim 31 wherein the binder includes hydroxypropyl methylcellulose and the disintegrant includes cross-linked polyvinylpyrrolidone.

36. (canceled)

37. The method of claim 7 comprising:
about 75% to about 98% microcrystalline cellulose;
about 1% to about 10% hydroxypropyl methylcellulose; and
about 1% to about 20% cross-linked polyvinylpyrrolidone.

38. The method of claim 7 comprising:
about 80% to about 90% microcrystalline cellulose;
about 2% to about 8% hydroxypropyl methylcellulose; and
about 3% to about 12% cross-linked polyvinylpyrrolidone.

39. The method of claim 7 comprising:
about 85% to about 93% microcrystalline cellulose;
about 2% to about 5% hydroxypropyl methylcellulose; and
about 10% cross-linked polyvinylpyrrolidone.

40. A pharmaceutical tablet comprising:
at least one active pharmaceutical ingredient; and
an excipient of substantially homogeneous, substantially spherical particles including:
a) microcrystalline cellulose;
b) at least one binder; and
c) at least one disintegrant.

41. The tablet of claim 40 wherein the at least one active pharmaceutical ingredient comprises about 1% to about 50% of the tablet.

42. The tablet of claim 40 further comprising a glidant, wherein the at least one active pharmaceutical ingredient comprises at least about 50% of the tablet, and wherein the tablet further comprises a glidant.

43. The tablet of claim 40 wherein the excipient includes:
about 75% to about 98% microcrystalline cellulose;
about 1% to about 10% at least one binder; and
about 1% to about 20% at least one disintegrant.

44. The tablet of claim 40 wherein the excipient includes:
about 80% to about 90% microcrystalline cellulose;
about 2% to about 8% at least one binder; and
about 3% to about 12% at least one disintegrant.

45. The tablet of claim 40 wherein the excipient includes:
about 85% to about 93% microcrystalline cellulose;
about 2% to about 5% at least one binder; and
about 10% at least one disintegrant.

46. The tablet of claim 40 wherein the binder includes hydroxypropyl methylcellulose and the disintegrant includes cross-linked polyvinylpyrrolidone.

47. The tablet of claim 40 wherein the active pharmaceutical ingredient includes ibuprofen.

48. The tablet of claim 40 wherein the active pharmaceutical ingredient includes atorvastatin calcium.

49. A method of making a pharmaceutical tablet comprising:
mixing at least one active pharmaceutical ingredient with an excipient of substantially homogeneous, substantially spherical particles according to claim 26; and
compressing the mixture to form a tablet.

50. The method of claim 49 wherein the tablet is formed by a rotary tabletting machine.

51. The method of claim 49 further including coating the tablet.
